SEQUENCE LISTING (4 sequences)

SEQ ID NO: 1 - protein, 75 aa, synthetic construct
  source 1..75: note = amino acid sequence of Dvl3-DEP peptide
PESGLEVRDRMWLKITIPNAFIGSDVVDWLYHNVEGFTDRREARKYASNLLKAGFIRHTVNKITFSEQCYYIFGD

SEQ ID NO: 2 - DNA, 225 bp, synthetic construct
  source 1..225: mol_type = other DNA; note = nucleotide sequence of Dvl3-DEP peptide
cccgagtcagggctggaggtccgagaccgcatgtggctcaagattaccattcccaatgctttcatcggctcagatgtggtggactggctgtatcacaatgtggaaggattcaccgaccggcgggaggcccgcaagtacgctagcaacctgctgaaggctggattcatccgccacacggtcaacaagatcacgttctctgagcagtgctactacatctttggtgac

SEQ ID NO: 3 - DNA, 32 bp, synthetic construct
  source 1..32: mol_type = other DNA; note = amplification primer for coding sequence of Dvl3-DEP peptide
ccgctcgagatgcccgagtcagggctggaggt

SEQ ID NO: 4 - DNA, 30 bp, synthetic construct
  source 1..30: mol_type = other DNA; note = amplification primer for coding sequence of Dvl3-DEP peptide
acgcgtcgacgtcaccaaagatgtagtagc

(19) United States
(12) Patent Application Publication
(10) Pub. No.: US 2024/0139284 A1
(43) Pub. Date: May 2, 2024

(54) METHOD FOR REPAIRING SERTOLI CELL INJURY IN TESTES

(71) Applicant: The Second Affiliated Hospital and Yuying Children's Hospital of Wenzhou Medical University, Wenzhou (CN)

(72) Inventors: Linxi Li, Wenzhou (CN); Lixiu Lv, Wenzhou (CN); Chuenyan Cheng, Wenzhou (CN); Jinjin Chu, Wenzhou (CN); Renshan Ge, Wenzhou (CN)

(21) Appl. No.: 18/320,895

(22) Filed: May 19, 2023

Related U.S. Application Data
(62) Division of application No. 17/541,211, filed on Dec. 2, 2021, now abandoned.

(30) Foreign Application Priority Data
Dec. 2, 2020 (CN) 202011398853.7

(51) Int. Cl. A61K 38/17; A61P 15/08; C12N 15/85

(52) U.S. Cl. CPC A61K 38/1709; A61P 15/08; C12N 15/85

(57) ABSTRACT

The present disclosure relates to use of a Dvl3-DEP peptide in the preparation of a medicament for repairing Sertoli cell injury in testes, and belongs to the technical field of medicine preparation. The Dvl3-DEP peptide is used in the preparation of a medicament for repairing Sertoli cell injury in testes, and the Dvl3-DEP peptide has an amino acid sequence shown in SEQ ID NO. 1. Tests prove that the overexpression of a Dvl3-DEP-coding gene significantly increases the level of cell resistance, repairs the tight junction function among Sertoli cells destructed by perfluorooctane sulphonate (PFOS) infection and enables a tight junction among Sertoli cells, and repairs the tight junction structure among Sertoli cells.

### CROSS REFERENCE TO RELATED APPLICATION(S)

This is a divisional of U.S. patent application Ser. No. 17/541,211 titled “USE OF Dvl3-DEP PEPTIDE IN PREPARATION OF MEDICAMENT FOR REPAIRING SERTOLI CELL INJURY IN TESTES,” filed on Dec. 2, 2021, which claims the benefit and priority of Chinese Patent Application No. 202011398853.7, filed on Dec. 2, 2020, the disclosure of which is incorporated by reference herein in its entirety as part of the present application.

### TECHNICAL FIELD

The present disclosure relates to the technical field of medicine preparation, and specifically to use of a Dvl3-DEP peptide in the preparation of a medicament for repairing Sertoli cell injury in testes.

### BACKGROUND

Perfluorooctane sulphonate (PFOS) is a degradation product of many different fluorinated organics in the environment. PFOS is widely present in the environment, including industrial production of textiles, paper, leather, daily chemical products and the like, which is difficult to decompose or degrade. PFOS can be enriched in organisms through food chains. PFOS taken by organisms through drinking water, food, or the like is difficult to be excreted by the organisms, which is finally enriched in organisms or various organs of the human body, with a human metabolic half-life of more than 5 years. A large number of studies have shown that PFOS has a variety of biological toxicities, including genotoxicity, neurotoxicity, developmental toxicity, reproductive toxicity, etc., and is considered to be an environmental pollutant with systemic multi-organ toxicity. PFOS exhibits a destructive effect on male reproductive functions, including: inducing testicular damage in rats, reducing the semen quality and testosterone level in males, or the like. At present, there are many reports about the treatment of PFOS-induced damage in reproductive functions, but there are no effective therapeutic medicaments available now.

### SUMMARY

The present disclosure is intended to provide use of a Dvl3-DEP peptide in the preparation of a medicament for repairing Sertoli cell injury in testes. Overexpression of Dvl3-DEP-coding gene can significantly increase the level of cell resistance and repair the tight junction function among Sertoli cells destructed by PFOS infection, the tight junction structure among Sertoli cells, and the cytoskeleton protein structure of Sertoli cells, thus achieving the repair of PFOS-induced Sertoli cell injury in testes.

The present disclosure provides use of a Dvl3-DEP peptide in the preparation of a medicament for repairing Sertoli cell injury in testes, and the Dvl3-DEP peptide has an amino acid sequence shown in SEQ ID NO. 1.

Preferably, a coding sequence of the Dvl3-DEP peptide may have a nucleotide sequence shown in SEQ ID NO. 2.

Preferably, the Sertoli cell injury in testes may include destruction to a tight junction function among Sertoli cells, injury to a tight junction structure among Sertoli cells, and reduction in a level of cytoskeleton protein polymerization in Sertoli cells.

Preferably, the Sertoli cell injury in testes may be caused by PFOS.

Preferably, a pair of amplification primers for the coding sequence of the Dvl3-DEP peptide may be shown in SEQ ID NO. 3 and SEQ ID NO. 4.

The present disclosure also provides a medicament for repairing Sertoli cell injury in testes, and the medicament includes a recombinant expression vector carrying a coding sequence of a Dvl3-DEP peptide.

Preferably, the recombinant expression vector may be based on pCI-neo.

Preferably, the coding sequence of the Dvl3-DEP peptide may be inserted to an XhoI/SalI multiple cloning site (MCS) of the pCI-neo.

Preferably, the medicament may be administered at an effective dosage: 5 μg to 50 μg of the recombinant expression vector per testis.

The present disclosure provides use of a Dvl3-DEP peptide in the preparation of a medicament for repairing Sertoli cell injury in testes. Test results show that, in DEP peptide-overexpressing cells, the level of cell resistance is significantly increased and the tight junction function among Sertoli cells destructed by PFOS infection is repaired; after Sertoli cells are infected with PFOS, the cell-cell junction interface is significantly damaged, showing discontinuity, shrinkage, void, and the like; in DEP peptide-overexpressing cells, the cell-cell junction is tight, and there is no obvious discontinuity, shrinkage, or the like, indicating that the overexpression of the DEP peptide repairs the tight junction structure among Sertoli cells; after PFOS treatment, a large number of polymerized F-actin and Tubulin microtubules are depolymerized to form free G-actin and Tubulin monomers, that is, Actin and Tubulin-based cytoskeletal structures are extensively destroyed; and in a DEP peptide-overexpressing Sertoli cell lysate system, there are many polymerized Actin and Tubulin components, indicating that overexpression of the DEP peptide can effectively prevent the depolymerization of these components and repairs the Actin and Tubulin-based cytoskeletal structures in Sertoli cells.

### BRIEF DESCRIPTION OF DRAWINGS

FIG. 1 is an electrophoretogram for a PCR amplification product of the DEP peptide-coding gene according to the present disclosure;

FIG. 2 is an electrophoretogram for digestion products obtained from double digestion of the pCI-neo-DEP mammalian overexpression vector according to the present disclosure;

FIG. 3 shows the resistance detection results for the permeability of the tight junction among Sertoli cells in vitro according to the present disclosure;

FIG. 4 shows the immunofluorescence (IF) staining results of tight junction-associated proteins in Sertoli cells in vitro according to the present disclosure;

FIG. 5 shows the Western blot results of polymerized/free Actin in Sertoli cells according to the present disclosure; and

FIG. 6 shows the Western blot results of polymerized/free Tubulin in Sertoli cells according to the present disclosure.

### DETAILED DESCRIPTION

The present disclosure provides use of a Dvl3-DEP peptide in the preparation of a medicament for repairing Sertoli cell injury in testes, and the Dvl3-DEP peptide has an amino acid sequence shown in SEQ ID NO. 1: PESGLEVRDRMWLKITIPNAFIGSDVVDWLYHNVEGFTDRREARKYASNLLKAGFIRHTVNKITFSEQCYYIFGD.

In the present disclosure, a coding sequence of the Dvl3-DEP peptide may have a nucleotide sequence shown in SEQ ID NO. 2: CCCGAGTCAGGGCTGGAGGTCCGAGACCGCATGTGGCTCAAGATTACCATTCCCAATGCTTTCATCGGCTCAGATGTGGTGGACTGGCTGTATCACAATGTGGAAGGATTCACCGACCGGCGGGAGGCCCGCAAGTACGCTAGCAACCTGCTGAAGGCTGGATTCATCCGCCACACGGTCAACAAGATCACGTTCTCTGAGCAGTGCTACTACATCTTTGGTGAC. In the present disclosure, the DEP peptide may function by overexpression in Sertoli cells. In the present disclosure, the coding sequence of the DEP peptide may preferably be present in the form of a recombinant expression vector. The recombinant expression vector may preferably be based on pCI-neo. In the present disclosure, the coding sequence of the DEP peptide may be introduced into Sertoli cells preferably by transfection. The present disclosure has no specific limitation on a method of the transfection, and a transfection method well known in the art may be adopted. In examples of the present disclosure, the transfection may be achieved by using a transfection reagent. The recombinant expression vector may be transfected at a dosage preferably of 5 μg/testis to 50 μg/testis, and more preferably of 15 μg/testis.

In the present disclosure, the Sertoli cell injury in testes may include destruction to a tight junction function among Sertoli cells, injury to a tight junction structure among Sertoli cells, and reduction in a level of cytoskeleton protein polymerization in Sertoli cells. Tests prove that the overexpression of a Dvl3-DEP-coding gene significantly increases the level of cell resistance, repairs the tight junction function among Sertoli cells destructed by PFOS infection and enables a tight junction among Sertoli cells, and repairs the tight junction structure among Sertoli cells. Moreover, after the overexpression of the Dvl3-DEP peptide, the level of cytoskeleton protein polymerization in the cells is significantly superior to that in an empty plasmid transfection group, and is comparable to that in a positive control group, indicating that the overexpression of the Dvl3-DEP peptide repairs the cytoskeleton structure of Sertoli cells.

In the present disclosure, the Sertoli cell injury in testes may be caused by PFOS. For example, after PFOS treatment, the Sertoli cell resistance is significantly decreased, and the cell-cell junction interface is significantly damaged, showing discontinuity, shrinkage, void, and the like. Moreover, PFOS infection can cause depolymerization of cytoskeleton proteins Actin and Tubulin in Sertoli cells, further impairing cell functions. The overexpression of the DEP peptide can significantly increase the level of cell resistance; repair the tight junction function among Sertoli cells destructed by PFOS infection and enable a tight junction among Sertoli cells; repair the tight junction structure among Sertoli cells; significantly alleviate the depolymerization of cytoskeleton proteins and repair the cytoskeleton protein structures of Sertoli cells to maintain the normal functions of the cytoskeleton proteins, thereby effectively maintaining the functions of Sertoli cells.

In the present disclosure, a pair of amplification primers for the coding sequence of the Dvl3-DEP peptide may be shown in SEQ ID NO. 3 (CCGCTCGAGATGCCCGAGTCAGGGCTGGAGGT) and SEQ ID NO. 4 (ACGCGTCGACGTCACCAAAGATGTAGTAGC).

The present disclosure also provides a medicament for repairing Sertoli cell injury in testes, and the medicament includes a recombinant expression vector carrying a coding sequence of a Dvl3-DEP peptide. In the present disclosure, the recombinant expression vector may be based on pCI-neo. In the present disclosure, the coding sequence of the Dvl3-DEP peptide may be inserted to an XhoI/SalI MCS of the pCI-neo. In the present disclosure, the medicament may be administered at an effective dosage: preferably 5 μg to 50 μg of the recombinant expression vector per testis; and more preferably 15 μg of the recombinant expression vector per testis.

The use of the Dvl3-DEP peptide in the preparation of a medicament for repairing Sertoli cell injury in testes according to the present disclosure will be further described in detail below with reference to specific examples. The technical solutions of the present disclosure include, but are not limited to, the following examples.

### Example 1

Cloning Process of a DEP Peptide-Coding Gene Fragment

Cloning primers of the DEP functional region were designed using SD rat testis cDNA with reference to a rat Dvl3-coding gene sequence NM 001107081.2 and a protein domain prediction of the website http://smart.embl-heidelberg.de/.

upstream primer:
(SEQ ID NO. 3)
CCGCTCGAGATGCCCGAGTCAGGGCTGGAGGT;
and
downstream primer:
(SEQ ID NO. 4)
ACGCGTCGACGTCACCAAAGATGTAGTAGC.

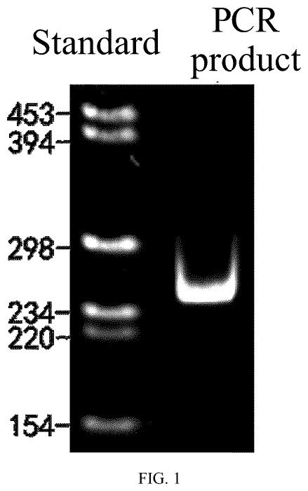

The primers included restriction endonuclease sites XhoI/SalI, start/stop codons, and protective bases. After PCR amplification, DEP peptide-coding gene fragments were obtained and detected by electrophoresis. The electrophoresis result is shown in FIG. 1, which is an electrophoretogram for a PCR amplification product of the DEP peptide-coding gene. It can be seen from FIG. 1 that the PCR product has a predicted length of 244 bp, and the electrophoresis result is consistent with the predicted product length. It can be known that a PCR product with the predicted length is obtained from amplification.

2. Sequencing of DEP Peptide-Coding Gene Fragments

The coding gene fragments obtained from PCR amplification were recovered and sent to GeneWiz for sequencing. A sequencing result is shown in SEQ ID NO. 2 (CCCGAGTCAGGGCTGGAGGTCCGAGACCGCATGTGGCTCAAGATTACCATTCCCAATGCTTTCATCGGCTCAGATGTGGTGGACTGGCTGTATCACAATGT GGAAGGATTCACCGACCGGCGGGAGGCCCGCAAGTACGCTAGCAACCTGCTGAAGGCTGGATTCATCCGCCACACGGTCAA CAAGATCACGTTCTCTGAGCAGTGCTACTACATCTTTGGTGAC).

The sequencing result shows a homology of 100% with a DEP domain-coding gene in the reference rat laminin α2-coding gene sequence NM 001107081.2, indicating that DEP peptide-coding gene fragments were successfully cloned.

3. Construction of a Recombinant Expression Vector for the DEP Peptide

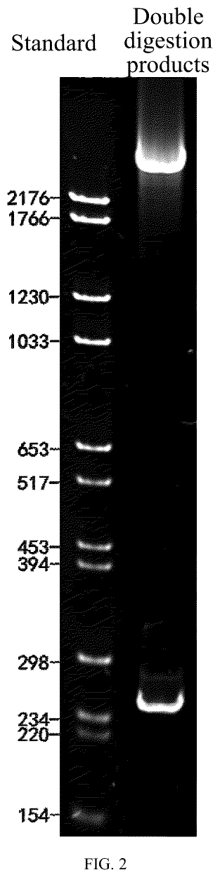

The cloned DEP peptide-coding gene fragment was ligated to a eukaryotic expression vector pCI-neo (Promega) to obtain an expression vector pCI-neo-DEP. The expression vector was double-digested with XhoI/SalI. FIG. 2 is an electrophoretogram for digestion products obtained from double digestion of the pCI-neo-DEP mammalian overexpression vector. It can be seen from FIG. 2 that the inserted fragment has a length of about 236 bp, and thus a correct expression vector is cloned.

### Example 2

Isolation and cultivation of primary Sertoli cells in testes

A total of 10 male SD rats at the age of 20 days were sacrificed and testes were collected. The testicular envelope and free seminiferous tubules were removed, and the remaining tissue was cut to 1 mm 3 with scissors and then digested successively with trypsin, protease inhibitor, collagenase, hyaluronidase, and other enzymes. Cell masses obtained from digestion were pipetted up and down with a glass Pasteur pipette and then subjected to density gradient centrifugation, and finally the volume of precipitated cells was measured and converted into the number of cells. Finally, the cells were cultivated in an F12/DMEM cell culture medium with human transferrin (5 μg/mL), bovine insulin (101 μg/mL), bacitracin (5 μg/mL), epithelial growth factor (EGF) (2.5 ng/mL), and the like. The cells were cultivated at 5% CO₂ and 35° C. to obtain primary Sertoli cells.

### Example 3

Repair of the DEP Peptide Overexpression in Sertoli Cells Infected with PFOS to the Junction Function Among Cells

The Sertoli cells isolated in Example 2 can form a tight junction morphology similar to that of epithelial cells under in vitro cultivation conditions. Matrigel diluted with the culture medium was coated on the surface of a nitrocellulose membrane of a cell culture chamber before the isolated primary Sertoli cells were inoculated at a density of 10×10 5 cells/cm 2. After the Sertoli cells were attached to the culture chamber 6 h to 8 h later, the cell resistance tester Millicell-ERS (Millipore) was used to detect the trans-epithelial electrical resistance (TEER) once a day. PFOS treatment was conducted on day 3 of Sertoli cell cultivation for a total of 24 h, with a final PFOS concentration of 20 μM. On day 4 (24 h after the PFOS treatment), the medium was removed, and the DEP peptide eukaryotic expression vector pCI-neo-DEP or empty vector pCI-neo was transfected using a transfection reagent of Lipojet In Vitro Transfection Reagent (SignaGen Laboratories, Rockville, MD, USA). The total transfection system was of 3 μl, including 1 μg of the recombinant expression vector DNA prepared in Example 1. 12 h after the transfection, the transfection reagent was washed away. The cell resistance was detected every day to day 7 after the isolation of the primary cells. Each group adopted 3 cell culture chambers.

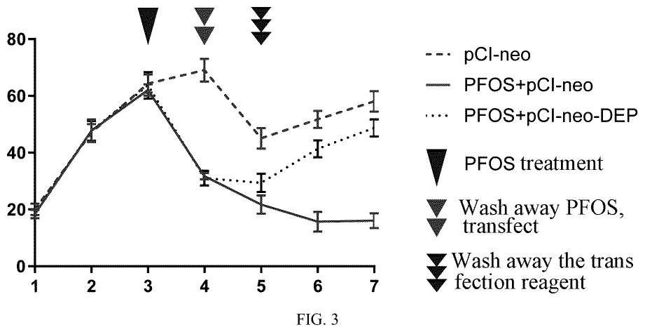

Results are shown in FIG. 3. FIG. 3 shows resistance detection results for the permeability of the tight junction among Sertoli cells in vitro. It can be seen from FIG. 3 that, after the PFOS treatment, the cell resistance was significantly reduced; and the cell resistance values of the two groups (empty plasmid control group and DEP peptide expression group) decreased by 35 Ohm·cm² to 40 Ohm·cm² compared with the blank control group, indicating that PFOS exhibited a significant destructive effect on the tight junction function among Sertoli cells. The blank control group and the empty plasmid control group were transfected with the pCI-neo empty plasmid, and the DEP peptide expression group was transfected with the pCI-neo-DEP plasmid. Because the transfection reagent had some toxicity, the cell resistance values of all groups were decreased to varying degrees. However, in the detection on day 6 of cultivation, it was found that the level of cell resistance in the group transfected with pCI-neo-DEP plasmid was significantly increased, and the increase continued to day 7 of cultivation. It showed that the overexpression of the DEP peptide repaired the tight junction function among Sertoli cells damaged by PFOS infection.

### Example 4

Morphological Observation of the Repair of DEP Peptide Overexpression in Sertoli Cells Infected with PFOS In Vitro to the Junction Function Among Cells

One day before isolation of Sertoli cells, sterilized cover glasses were placed in a 12-well plate, and the surface was coated with Matrigel diluted with the cell culture medium. The plate was aseptically air-dried and placed in a cell incubator for later use. Sertoli cells were isolated and then inoculated into the 12-well plate for cultivation, with an inoculation density of 40,000 cells/cm². PFOS treatment was conducted on day 3 of cultivation. PFOS was added at a final concentration of 20 μM, and 24 h after the treatment (day 4 of cultivation), PFOS was washed away. The transfection reagent and the pCI-neo empty plasmid or pCI-neo-DEP expression plasmid corresponding to a respective group were simultaneously added. After the transfection was conducted for 12 h, the transfection system was washed away and fresh culture medium was added. The cultivation was continued to day 6 after the isolation, and the cover glasses inoculated with cells were collected, and then a paraformaldehyde (PFA) solution was added for fixing.

In order to observe the tight junction morphology among Sertoli cells cultivated in vitro, IF staining (green fluorescent) was conducted using an antibody for the Sertoli cell tight junction-associated protein ZO-1 (ThermoFisher, 61-7300), and the nucleus was stained with DAPI (blue fluorescence) after the above staining. After mounting, a fluorescence microscope was used to observe and take pictures.

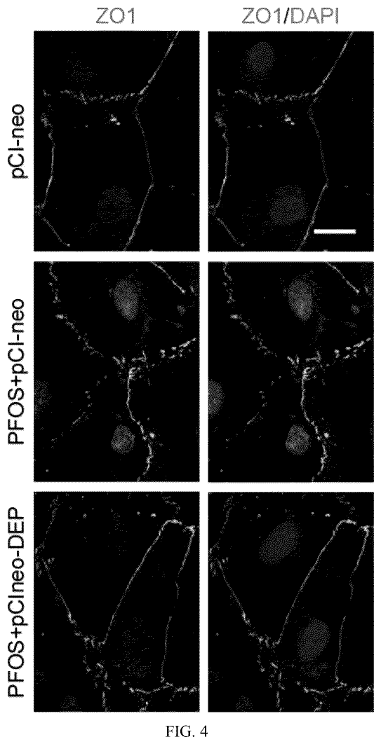

Experimental results are shown in FIG. 4. It can be seen that a cell-cell junction interface was significantly damaged after the Sertoli cells were infected with PFOS, and compared with the blank control group, in the empty plasmid transfection group, the cell-cell junction interface underwent discontinuity, shrinkage, void, and the like due to PFOS infection. It showed that the tight junction structure formed by Sertoli cells in vitro was damaged by PFOS induction. However, the cell-cell junction in the DEP peptide overexpression group was similar to that in the blank control group, which was tight and had no obvious discontinuity, shrinkage, or the like. Results showed that the overexpression of DEP peptide repaired the tight junction structure of Sertoli cells.

### Example 5

Repair of the DEP Peptide Overexpression in In Vitro Sertoli Cells Infected with PFOS to the Polymerization Ability of Cytoskeleton Proteins Actin and Tubulin

Isolation and cultivation of cells were conducted according to the method in Example 2, and obtained cells were inoculated in a prepared 6-well plate. Cells in each group were collected immediately after the experiment ended. Related Actin and Tubulin polymerization tests were conducted on cells in the DEP peptide overexpression group and cells in the empty plasmid transfection group using G-Actin/F-actin polymerization detection kit (G-Actin/F-actin In Vivo Assay Biochem Kit, BK037, Cytoskeleton, Inc) and Microtubule/Tubulin polymerization detection kit (Microtubule/Tubulin In Vivo Assay Biochem Kit, BK038, Cytoskeleton, Inc). Related detection methods can refer to product descriptions. The main purpose of these tests is to separate polymerized cytoskeleton proteins (F-actin and Microtubule) and free monomeric cytoskeleton proteins (G-actin and Tubulin) in a cell lysate by ultracentrifugation. Then the polymerized cytoskeleton protein (a precipitate obtained from the ultracentrifugation) and the monomeric cytoskeleton protein (a supernatant obtained from the ultracentrifugation) in the cell lysate system were detected separately by Western blot. For the Actin polymerization test, a cell lysate treated with 0.1 μM phalloidin was adopted as a positive control, because phalloidin could promote the polymerization of all Actin monomers in the system; and a cell lysate treated with 80 mM urea was adopted as a negative control, because urea could promote the depolymerization of all polymerized Actin in the system. For Tubulin polymerization test, a cell lysate treated with 20 μM paclitaxel was adopted as a positive control, because paclitaxel could promote the polymerization of all Tubulin monomers in the system; and a cell lysate treated with 2 mM calcium chloride was adopted as a negative control, because calcium chloride could promote the depolymerization of all polymerized Microtubule in the system. The cell lysates of the above control groups were from untreated Sertoli cells.

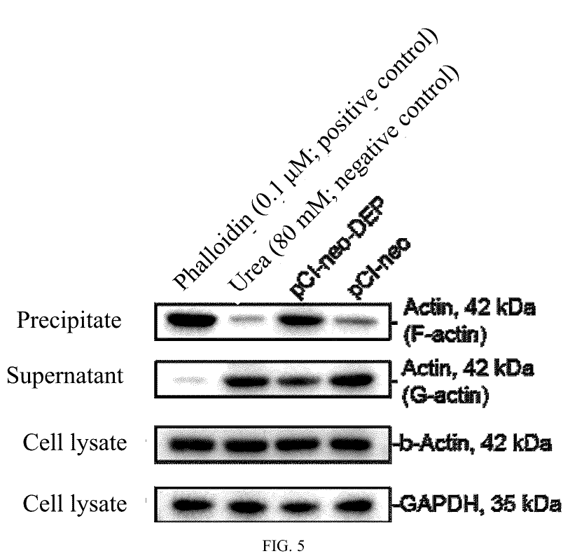

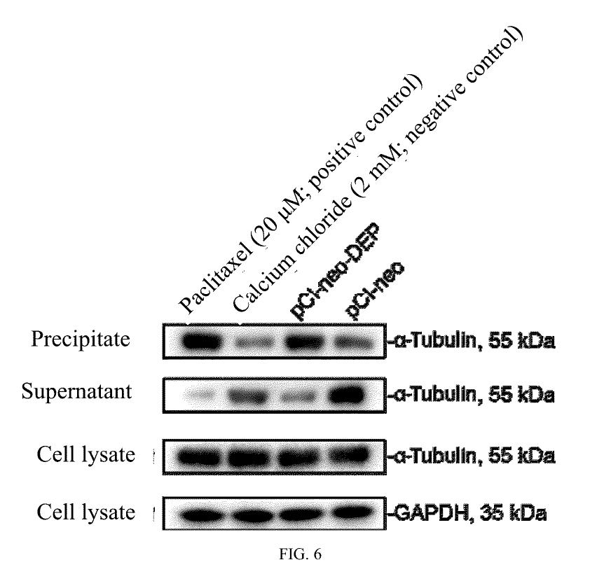

Experimental results are shown in FIG. 5 and FIG. 6.

FIG. 5 shows the Western blot results of polymerized/free Actin in Sertoli cells. The cell lysate in each group was subjected to ultracentrifugation, and then a resulting precipitate was subjected to Western blot. Western blot results showed that, after the PFOS treatment, the F-actin level in the pCI-neo empty plasmid transfection group was low, which was almost comparable to that in the negative control group, indicating that a large amount of depolymerization occurred in the F-actin cytoskeleton structure in PFOS-treated cells. The F-actin level in the DEP peptide overexpression group was relatively high, which was lower than that in the positive control group but significantly higher than that in the empty plasmid transfection group. It showed that the overexpression of DEP could repair the depolymerization of the F-actin cytoskeleton structure in Sertoli cells induced by PFOS infection, thus promoting the polymerization of Actin. Moreover, the Western blot result of the supernatant obtained from the ultracentrifugation also showed that, after the PFOS treatment, the free G-actin content in the cell lysate supernatant of the empty plasmid transfection group was higher than that of the DEP peptide overexpression group, further proving that DEP exhibited a repair effect on the F-actin cytoskeleton structure. FIG. 6 shows the Western blot results of polymerized/free Tubulin in Sertoli cells. The cell lysate in each group was subjected to ultracentrifugation, and then a resulting precipitate was subjected to Western blot. Western blot results showed that, after the PFOS treatment, the Microtubule level in the pCI-neo empty plasmid transfection group was low, which was almost comparable to that in the negative control group, indicating that a large amount of depolymerization occurred in the Microtubule cytoskeleton structure in PFOS-treated cells. The Microtubule level in the DEP peptide overexpression group was relatively high, which was lower than that in the positive control group but significantly higher than that in the empty plasmid transfection group. It showed that the overexpression of DEP could repair the depolymerization of the Microtubule cytoskeleton structure in Sertoli cells induced by PFOS infection, thus promoting the polymerization of Tubulin. The above results indicated that the overexpression of the DEP peptide significantly alleviated the depolymerization of cytoskeleton proteins, maintained the normal functions of cytoskeleton proteins, and thus effectively maintained the functions of Sertoli cells.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. Use of a Dvl3-DEP peptide in the preparation of a medicament for repairing Sertoli cell injury in testes, wherein, the Dvl3-DEP peptide has an amino acid sequence shown in SEQ ID NO. 1.

2. The use according to claim 1, wherein, a coding sequence of the Dvl3-DEP peptide has a nucleotide sequence shown in SEQ ID NO. 2.

3. The use according to claim 1, wherein, the Sertoli cell injury in testes comprises destruction to a tight junction function among Sertoli cells, injury to a tight junction structure among Sertoli cells, and reduction in a level of cytoskeleton protein polymerization in Sertoli cells.

4. The use according to claim 1, wherein, the Sertoli cell injury in testes is caused by perfluorooctane sulphonate (PFOS).

5. The use according to claim 1, wherein, a pair of amplification primers for the coding sequence of the Dvl3-DEP peptide are shown in SEQ ID NO. 3 and SEQ ID NO. 4.

6. A medicament for repairing Sertoli cell injury in testes, wherein, the medicament comprises a recombinant expression vector carrying a coding sequence of a Dvl3-DEP peptide.

7. The medicament according to claim 6, wherein, the recombinant expression vector is based on pCI-neo.

8. The medicament according to claim 7, wherein, the coding sequence of the Dvl3-DEP peptide is inserted to an XhoI/SalI multiple cloning site (MCS) of the pCI-neo.

9. The medicament according to claim 6, wherein, the medicament is administered at an effective dosage: 5 μg to 50 μg of the recombinant expression vector per testis.
